(19) United States
(12) Patent Application Publication
(10) Pub. No.: US 2024/0075224 A1
(43) Pub. Date: Mar. 7, 2024

(54) CONNECTOR FOR A PATIENT VENTILATION SYSTEM

(71) Applicant: RAUMEDIC AG, Münchberg (DE)

(72) Inventors: Thomas RUHLAND, Bayreuth (DE); Ricardo EHRENPFORDT, Zwickau (DE); Stefan SEUFERLING, Hilpoltstein (DE)

(73) Assignee: RAUMEDIC AG, Münchberg (DE)

(21) Appl. No.: 17/767,395

(22) PCT Filed: Oct. 8, 2020
(86) PCT No.: PCT/EP2020/078220

§ 371 Date: Apr. 7, 2022

(30) Foreign Application Priority Data
Oct. 9, 2019 (DE) 10 2019 215 483.2
Oct. 25, 2019 (DE) 10 2019 216 487.0

(51) Int. Cl. A61M 16/00; A61M 16/08

(52) U.S. Cl. CPC A61M 16/0051; A61M 16/0816; A61M 16/0875; A61M 2205/584; A61M 2230/50

(57) ABSTRACT

A connector for a patient ventilation system serves for connecting to a heatable breathing air tube portion for conducting ventilation air from a breathing air source to a patient and for connecting the breathing air tube portion to a patient air interface. At least one portion of the connector is formed of a thermochromic material. This results in a connector in which a temperature of the heatable breathing air tube portion is reliably displayed.

### CROSS-REFERENCES TO RELATED APPLICATIONS

This application claims the priorities of German Patent Application, Serial No. DE 10 2019 215 483.2, filed Oct. 9, 2019 and DE 10 2019 216 487.0, filed on Oct. 25, 2019, the content of which is incorporated herein by reference in its entirety as if fully set forth herein.

### FIELD OF THE INVENTION

The invention relates to a connector for a patient ventilation system.

### BACKGROUND OF THE INVENTION

Such a connector is known from DE 20 2014 103 998 U1. US 2012/0125333 A1 discloses a ventilation system. GB 2572468 A discloses a safety warning system which can be used with a ventilation circuit. US 2012/0136267 A1 discloses a device and a method for visibly displaying whether ventilation is taking place.

### SUMMARY OF THE INVENTION

It is an object of the present invention to further develop a connector for a patient ventilation system which is flexible with regard to its possible applications in such a way that a temperature of the heatable breathing air tube portion is reliably displayed.

This object is achieved according to the invention by a connector for a patient ventilation system
to connect to a heatable breathing air tube portion for conducting ventilation air from a breathing air source to a patient,
to connect the breathing air tube portion to a patient air interface,
wherein at least one portion of the connector is made of a thermochromic material,
wherein the thermochromic material includes a plastic matrix having a poly(organo)siloxane as well as by a connector for a patient ventilation system
to connect to a heatable breathing air tube portion for conducting ventilation air from a breathing air source to a patient,
to connect the breathing air tube portion to a patient air interface,
wherein at least one portion of the connector is made of a thermochromic material,
wherein the connector has a plurality of portions which are each made of a thermochromic material having different color change temperatures.

According to the invention, it has been found that the use of a thermochromic material leads to the simple possibility of displaying the temperature of the corresponding connector portion and, through this, the temperature of the conducted breathing air. An additional display can be omitted. Complex electrical connections or electrical components as well as a cone-sponding sensor system communicating therewith can also be dispensed with.

In the simplest embodiment, the connector may be a breathing air tube portion itself.

The thermochromic material may have a plastic matrix. This plastic matrix can be a thermoplastic elastomer.

According to a first aspect, the plastic matrix of the thermochromic material may have a poly(organo)siloxane. Thus, a silicone-based thermochromic material may be used. This ensures good biocompatibility of the thermochromic material.

The plastic matrix of the thermochromic material can be made entirely of silicone. An entire component of the connector or, if necessary, the entire connector can be made of thermochromic material. Alternatively, the thermochromic material can be inserted into the connector, for example as a strip.

Other materials or material compositions known in connection with thermochromic material can also be used.

According to a further aspect, a plurality of portions of the connector are each formed of a thermochromic material having different color change temperatures. Such an embodiment of the connector makes it possible not only to indicate whether a certain color change temperature has been reached or exceeded, but also to provide an absolute temperature indication within predefinable error limits Due to the multiple structure realized over the plurality of portions of the connector made of thermochromic material having different color change temperature, a plurality of temperature thresholds can be monitored. A temperature range can be monitored. A number of the portions of the connector, each formed from a thermochromic material having a different color change temperature, may specify a temperature resolution. For example, a temperature range between 38° C. and 42° C. can be monitored with five portions whose color change temperature differs by 1 K each.

In addition to the portions having the different color change temperatures, a temperature scale can also be arranged on the connector. A legend that assigns temperatures or product information to the various colors can also be applied directly to the connector.

A design of the connector in which at least one of the portions is made of a reversible thermochromic material and that at least another one of the portions is made of a non-reversible thermochromic material ensures, for example, to permanently record a certain temperature overshoot and/or a certain temperature undershoot. An exceedance of a temperature threshold may be stored by the portion made of non-reversible thermochromic material. An additionally possible portion of the connector made of a reversible thermochromic material may be used to monitor a current temperature value of the connector.

In the case of a continuous color progression of the reversible/non-reversible portion of the connector made of thermochromic material as a function of temperature, the respective connector temperature can be read from a color scale.

This makes it possible to assess whether a certain operating state has oc-curred over a longer period of time, e.g. a patient's fever attack or a failure of a heating unit.

The advantages of an assembly having at least one connector according to the invention, and a breathing air tube component for conducting breathing air correspond to those already explained above with reference to the connector.

In an assembly in which at least one tube portion of the breathing air tube component is in contact with or thermally coupled to the ventilation air, wherein the tube section is made of a thermochromic material, furthermore, the temperature of the breathing air conducted in the tube portion can be thermally monitored via the thermochromic material. For the possible de-signs of the thermochromic material portion, what has been stated above regarding the corresponding thermochromic material portion of the connector applies. In principle, the connector itself can also be designed without a portion of thermochromic material in such an assembly.

The features of the connectors of the various solution aspects discussed above may be present in any combination.

Examples of embodiments of the invention are explained in more detail below with reference to the drawing.

### BRIEF DESCRIPTION OF THE DRAWINGS

FIG. 1 schematically shows main components of a patient ventilation system including a main unit, a humidification device, a breathing mask, and a plurality of heatable tube components connecting them and conducting the breathing air, including a plurality of connectors for the fluid-conducting connection of these components;

FIG. 2 in perspective shows an end portion of one of the tube components with a junction connector and a monitoring connector shown spaced apart from it, i.e. not yet in the connecting position,

FIG. 3 shows another perspective view of the monitoring connector;

FIG. 4 shows an exploded view illustrating a reusable component of the monitoring connector separate from a disposable component of the monitoring connector;

FIG. 5 shows another exploded view of the monitoring connector, wherein a printed circuit board of the disposable component as a component of a start signal generator unit and a cover therefor are additionally shown, which are used to generate a start signal for detecting a duration of use of the disposable component;

FIG. 6 shows an axial section through the monitoring connector partially revealing internal details, wherein in particular components of a control/regulation unit, a light source and a start signal generator unit are shown;

FIG. 7 shows a perspective of a further embodiment of a monitoring connector similar to FIG. 2, which at the same time has the function of a junction connector comparable to the embodiment shown in FIG. 2;

FIG. 8 shows another embodiment of a disposable component of the monitoring connector having a connector portion made of a thermochromic material; and

FIG. 9 shows a portion of an embodiment of a tube component of the ventilation system having a wall portion made of a thermochromic material.

### DESCRIPTION OF THE PREFERRED EMBODIMENTS

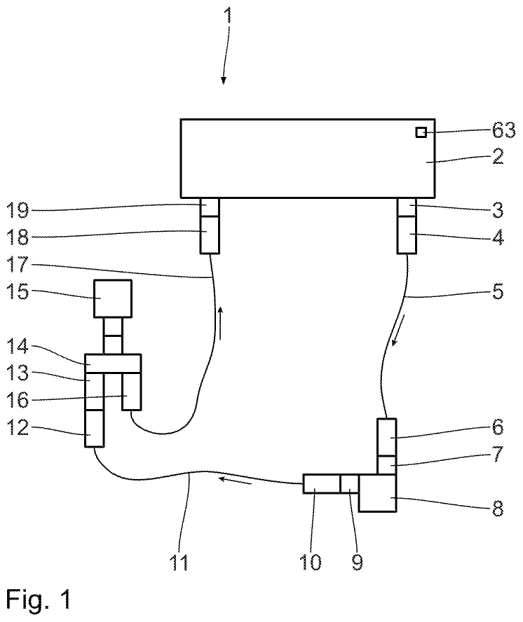

A patient ventilation system 1, the main components of which are shown in FIG. 1, serves for the ventilation of a patient in a clinical, other inpatient or even home care setting.

The essential, air-conducting components of the ventilation system 1 are made of plastic material.

The ventilation system has a main unit 2 for controlling/regulating, in particular, a breathing air supply, air humidification and temperature control of the breathing air. In principle, the main unit 2 may also serve to deter-mine a breathing air composition. The main unit 2 serves as a breathing air source.

The main unit 2 is in fluid connection with a breathing air supply tube component 5 via a junction port 3 and a junction connector 4. The latter is shown very schematically in FIG. 1, as are the other tube components. The tube components of the ventilation system 1 can be heated in a con-trolled/regulated manner via the main unit 2, as will be described below.

Via a further junction connector 6, the breathing air supply tube component is in fluid connection with a junction port 7 of a breathing air humidification device 8. The humidification device 8 is in signal communication with the main unit 2 in a manner not shown.

The humidification device 8 is in fluid connection with a breathing air connection tube component 11 via a further junction port 9 and a further junction connector 10.

The breathing air connection tube component 11 is in fluid connection with a patient breathing mask 15 via a further junction connector 12, a monitoring connector 13 and a three-way connector 14. The monitoring connector 13 thus, on the one hand, serves to connect to the breathing air connection tube component 11, i.e. to a heatable breathing air tube portion for conducting ventilation air from the main unit 2 to the patient, and, on the other hand, to connect this tube component 11 to the patient breathing mask 15, as a patient air interface.

In a variant not shown in more detail, the monitoring connector 13 is integrally connected to the tube component 11, i.e. forms an integral part therewith.

The breathing mask 15, via the three-way connector 14 and a further junction connector 16, is in fluid connection with a breathing air discharge tube component 17. The latter then is in fluid connection with the main unit 2 via a further junction connector 18 and a further junction port 19.

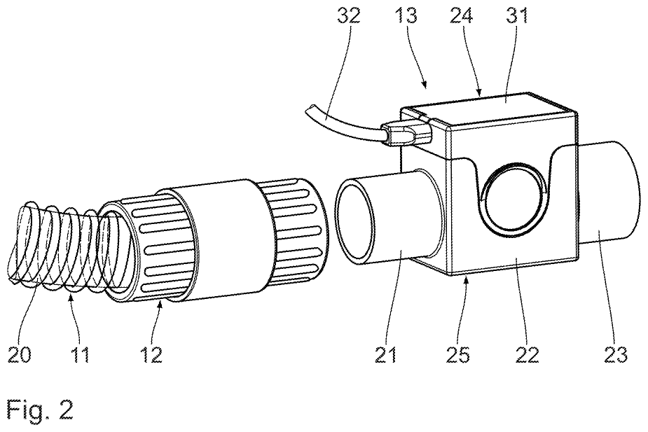

FIG. 2 shows a portion of the breathing air connection tube component 11 together with the junction connector 12 and, shown separately therefrom, the monitoring connector 13. The tube component 11 has an outer heating coil 20, embodied as an electrical resistance heater, for heating the breathing air conducted in the tube component 11. The junction connector 12 has a receptacle, not visible in FIG. 2, that is designed to be complementary to a supply junction sleeve 21 of the monitoring connector 13. The supply junction sleeve 21 is formed on a base body 22 of the monitoring connector. Opposite to the supply junction sleeve 21, a discharge junction sleeve 23 of the monitoring connector 13 is formed on the base body 22. An outer diameter of the supply junction sleeve 21 is smaller than an outer diameter of the discharge junction sleeve 23. A flow of breathing air through the monitoring connector 13 is ensured via the two junction sleeves 21, 23 and an opening in the base body 22 that is aligned with them.

In an embodiment not shown, the supply junction sleeve 21 and also the junction connector 12 are omitted and the base body 22 of the monitoring connector 13 is formed directly on the tube component 11 so that an air conduction connector component of the monitoring connector 13, that is, the base body 22, forms an integral part with the breathing air tube component 11.

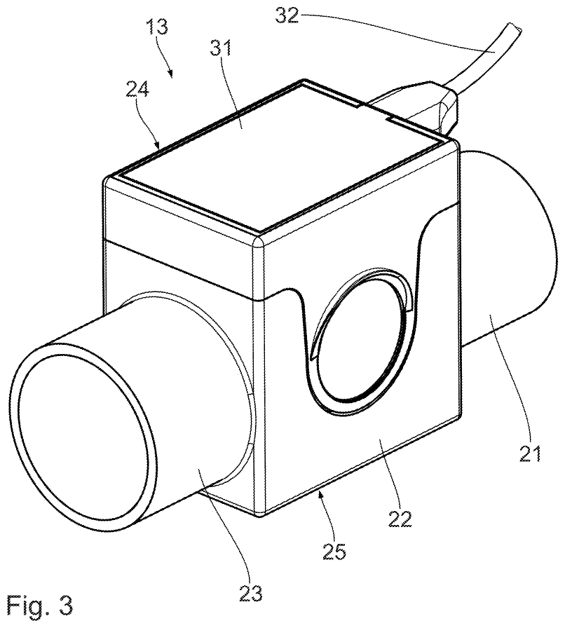

FIGS. 3 to 6 show further details of the monitoring connector 13.

The monitoring connector 13, as its main components, has a reusable component 24, also referred to as reusable, and a disposable component 25, also referred to as disposable. In principle, it is also possible to use the disposable 25 multiple times after appropriate cleaning or sterilization.

The reusable 24 is detachably linked to the disposable 25 via an electrical plug connection 26. The plug connection has multiple poles and eight poles in the illustrated embodiment. In the embodiment shown, a plug 27 of the plug connection 26 is designed as part of the disposable 25.

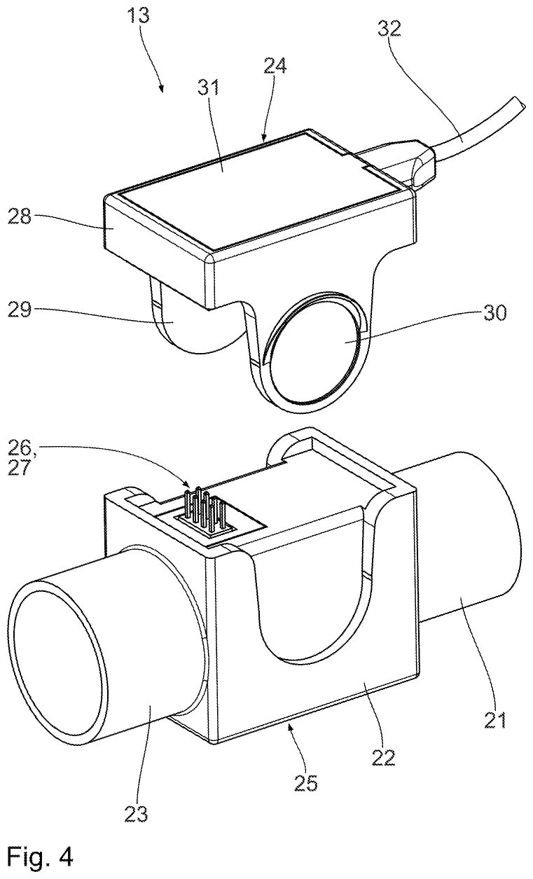

The reusable 24 has a cuboid base body 28 from which two projection components 29, 30 extend away, which in the assembled state with the disposable 25 cover two opposite side walls of the disposable 25 in portions. With the exception of a cover 31 which closes the base body 28 upwardly in the orientation according to FIG. 4, visible sides of the base body 28 are made of transparent or opaque material. The base body 28 additionally has a supply plug connection for connecting a supply line 32, via which an electrical supply and/or a signal transmission from/to an external component, in particular the main unit 2, is possible.

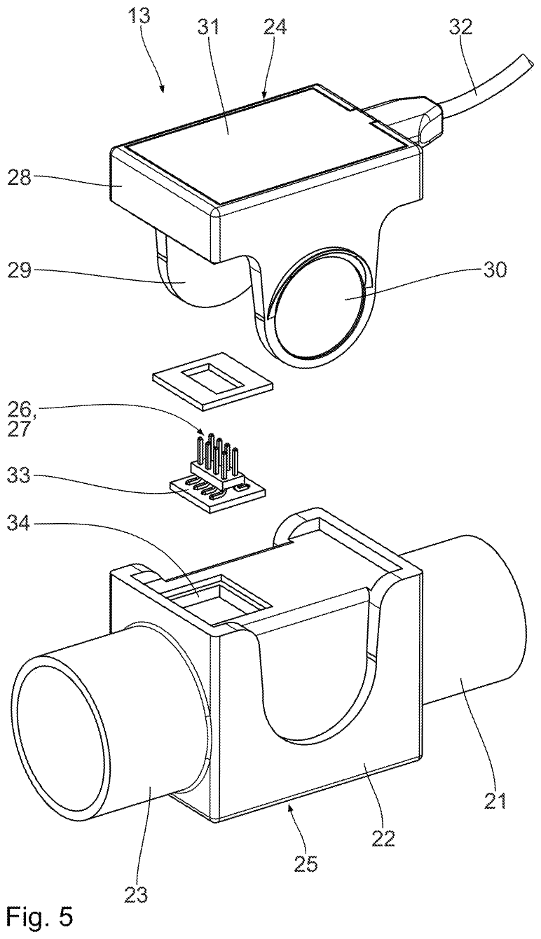

FIG. 5 shows in an additional exploded view an embedding of a printed circuit board 33 in a corresponding receiving space 34 in the base body 22 of the disposable 25. The receiving space 34 is in communication via a coupling wall, via a window or via a passage opening with an inner lumen of the base body 22 linking the two junction sleeves 21, 23. This can be used for a sensory coupling of a sensor of the disposable 25, accommodated in the receiving space 34, to the breathing air conducted therethrough.

The monitoring connector 13 has a control/regulation unit 35, which is accommodated in a receiving space 36 of the reusable 24. The core component of the control/regulation unit 35 is a microcontroller 37, which is accommodated together with other components on a printed circuit board 38 in the receiving space 36. The control/regulation unit 35 further includes a real time clock 39, which may include a quartz crystal timer, and a connection controller 40, which is also embodied as a microcontroller and controls a data connection between the components on the printed circuit board 38 of the reusable 24 and the components on the printed circuit board 33 of the disposable 25. A sensor controller 41 may be provided on the printed circuit board 38 as an additional component of the control/regulation unit 35. The microcontroller 37 and the real-time clock 39 are located on one side of the printed circuit board 38, and the connection controller 40 and the sensor controller 41 are located on the opposite side of the printed circuit board 38, on which a socket of the plug connection 26 is also arranged.

The electronic components arranged on the printed circuit boards 33 and 38, respectively, may be embodied as SMD components. The printed circuit boards 33, 38 may be double-layer PCBs. An internal communication between the components on the printed circuit boards 33, 38 may take place via an I²C interface standard.

Furthermore, two light sources 42 are arranged on the printed circuit board 38, which are designed as RGB LEDs and are in signal communication with the control/regulation unit 35. Depending on the control via the microcontroller 37, the respective light source 42 can therefore emit, for example, red light, green light, blue light or also white light. This emitted light is visible through the transparent/opaque portions of the base body 28 of the reusable 24.

The number of light sources 42 may vary from 1 to 10, depending on the design of the monitoring connector 13.

The respective light source 42 thus has a plurality of individual light sources of different colors via these RGB LEDs. Controlled via the microcontroller 37, each of these colors is assigned to a state of the monitoring connector 13 or a state of the ventilation system 1. In addition, the microcontroller 37 can specify an activation frequency of the light source 42 so that, for example, further states of the monitoring connector 13 or of the ventilation system 1 can be indicated via a flashing sequence of the light source 42.

By means of a light guidance of the light emitted by the light sources 42 via the transparent/opaque portions of the base body 28 as well as the projection components 29, 30, it is ensured that the light signal generated by the light sources 42 is visible from at least five spatial directions. Visibility directly from above, i.e. from a viewing direction perpendicular to the plane of arrangement of the cover 31, is provided by the fact that the trans-parent/opaque base body 28 is designed to project over the entire circum-ference of the cover 31. The other four spatial directions from which a light signal of the light sources 42 are visible are the main directions associated with the four side walls of the base body 28.

A memory component 43 is also arranged on the printed circuit board 33 of the disposable 25. As ROM data, the memory component contains an identification data set that uniquely identifies the disposable 25. The identification signal may be, for example, an individual identification number of the disposable 25, which is assigned when the disposable 25 is manufactured.

Together with the microcontroller 37 and, if applicable, the connection controller 40, the memory component 43 constitutes a start signal generator unit 44 for generating a start signal from which the control/regulation unit detects a duration of use of the disposable 25. For this purpose, a transmission of the identification data set between the memory component 43 and the microcontroller 37 generates the start signal. Alternatively or additionally, the start signal generator unit may have only the microcontroller 37 and, if applicable, the connection controller 40, and may be designed such that establishing an electrical contact between the reusable 24 and the disposable 25 via the plug connection 26 generates the start signal. A trig-ger by the main unit 2 may also generate the start signal via an appropriate control signal communication.

The printed circuit board 33 of the disposable 25 further carries a sensor 45 for capturing a breathing air parameter. The sensor 45 is in signal communication with the microcontroller 37 of the control/regulation unit 35 via the plug connection 26. The sensor 45 is a temperature sensor. This latter may be operable in a temperature range between −20° C. and 90° C. and may, for example, have a measurement accuracy of 0.2 K in a range between −10° C. and 80° C. Signal data of the sensor 45 may be stored at least tempo-rarily in the memory component 43 of the disposable 25 and/or in a memory of the microcontroller 37. The microcontroller 37 and the memory component 43 can thus have the function of a signal data memory.

The microcontroller 37 includes a processing module 37a for processing signal data.

In turn, the signal data memory of the microcontroller 37 may include an identification data set as a ROM data set by which the reusable 24 can be uniquely identified.

The sensor 45 is in sensory communication, in particular in thermal contact, with the inner lumen 48 of the disposable 25 via a coupling medium 46 and a thin sensor wall portion 47. Accordingly, the coupling medium 46 is a material with very good thermal conductivity.

A wall thickness of the sensor wall portion 47 may be less than 1 mm, may be less than 0.5 mm, may be less than 0.25 mm, and may be less than 0.2 mm. As a rule, the wall thickness of the sensor wall portion 47 is greater than 25 μm.

The sensor 45 is designed such as to measure the breathing air parameter, i.e. the breathing air temperature, without contact, i.e. without direct contact with the breathing air.

The coupling medium 46, on the one hand, and the sensor wall portion 47, on the other hand, constitute a cover layer over which the sensor 45 is cov-ered towards the lumen 48 of the disposable 25, i.e. towards a breathing air conducting lumen.

As an alternative to a coupling medium and/or a wall cut, as a cover layer 46 a window may also be inserted into the base body 22 of the disposable 25, through which the sensor 45 is in sensory contact with the lumen 48 and thus with the breathing air.

The sensor 45 may then be an optical sensor and, in particular, a spatially resolving optical sensor.

In an alternatively or additionally possible sensor 45a, which is also carried by the printed circuit board 33, the sensor 45a measures the respective breathing air parameter in contact with the breathing air. For this purpose, instead of the sensor wall portion 47, a sensor window or a sensor recess 22a is embodied in the base body 22 of the disposable 25, via which a sensor surface of the sensor 45a comes into direct contact with the breathing air in the lumen 48. The sensitive sensor surface of the sensor 45a is arranged such that there is no interfering dead volume between it and the lumen 48. Preferably, the sensitive sensor surface of the sensor 45a is flush with a duct wall of the sensor window or sensor recess 22a in the lumen 48.

Such a sensor 45a which measures the breathing air parameter in direct contact with the breathing air may be, for example, an analysis sensor for determining a composition of the breathing gas, a sensor for measuring a flow rate of the breathing air, or a humidity sensor for the breathing air.

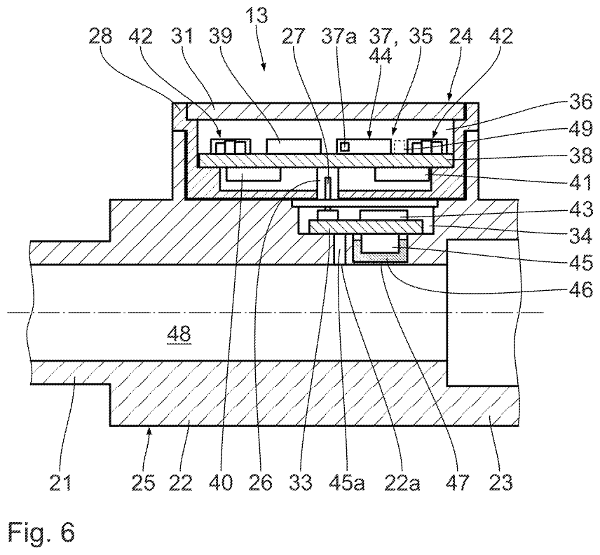

The monitoring connector 13 may additionally include an environmental sensor for capturing an environmental parameter, which is in signal communication with the control/regulation unit 35. For example, such a sensor may also be disposed on the printed circuit board 38, which is indicated at 49 in FIG. 6. The environmental sensor may be in sensor contact with the environment via a thin sensor wall portion of the base body 28 or via a window formed herein, similar to what has been described above with reference to the sensor 45. The environmental sensor 49 may have a micro-phone. The environmental sensor 49 may be configured as a motion detection sensor for detecting movement of the monitoring connector 13, for example as an acceleration sensor.

RS232 and/or RS485 can be used as interface standards.

A plurality of signal transmission interfaces of the control/regulation unit are implemented via the plug connection 26 as well as the connection controller 40. Thus, signal transmission is possible according to a plurality of and in particular different interface standards. An internal communication between the components on the printed circuit boards 33, 38 can be realized via an I²C interface standard. At least one of these interfaces is designed for signal communication with the sensor 45 and is linked to it accordingly. Another one of these interfaces is prepared for signal communication with a sensor not yet installed in this monitoring connector 13. In this way, expandability of the monitoring connector by additional sensors that work with other interface standards than the sensor 45 can be ensured.

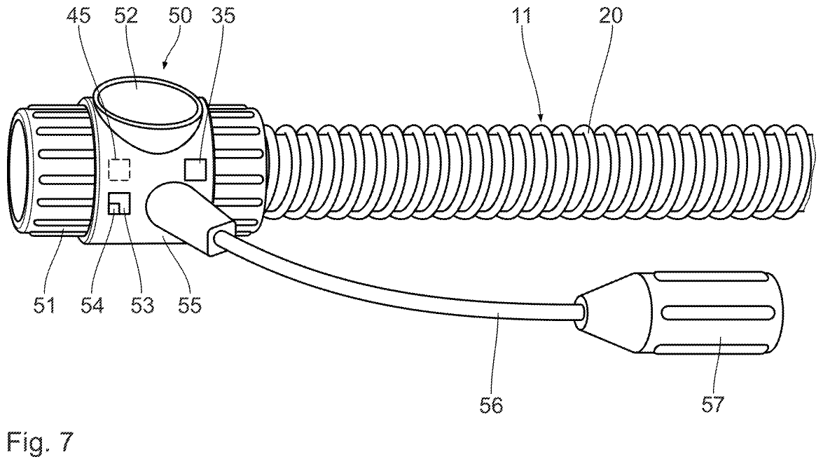

With reference to FIG. 7, a further embodiment of a monitoring connector 50 for the patient ventilation system is explained below. Components and functions corresponding to those already explained above with reference to FIGS. 1 to 6 bear the same reference numerals and are not discussed again in detail.

The monitoring connector 50 integrates the functions of the junction connector 12 and the monitoring connector 13 of the embodiment according to FIG. 2. Thus, on the one hand, the monitoring connector 50 serves for the mechanical and breathing air conducting connection of the breathing air connection tube component 11 to the three-way connector 14 and, on the other hand, has the control/regulation as well as monitoring functions of the monitoring connector 13, as explained above. The monitoring connector 50 is designed as a connector which links the tube component 11 to the patient breathing mask 15, i.e. the patient interface. Further, the monitoring connector 50 has at least one basic sensor in the manner of sensor 45 for capturing a breathing air measurement parameter. The basic sensor 45 is again indicated by dashed lines in FIG. 7. The monitoring connector 50 is integrally joined with the tube component 11 and, in particular, molded thereto.

An air-conducting connector component of the monitoring connector 50, that is, the base body 51 defining the inner lumen 48, forms an integral component with an inner lumen of the tube component 11. The base body 51 of the monitoring connector 50 may include the sensor in the manner of the sensor 45. An electronic connector component 52 of the monitoring connector 50, which has the control/regulation unit 35, is detachably connected to the base body 51 of the monitoring connector 50, for example via a mechanical plug connection. Furthermore, the monitoring connector 50 may have an additional sensor connector component 53 that includes an expansion sensor 54 for capturing another breathing air parameter, i.e., a different parameter than detected by the basic sensor.

An RFID chip, in particular for transmitting an identification data set, may be part of the electronic connector component 52.

A supply line 56 in the manner of the supply line 32 is linked to the monitoring connector 50 via a linking portion 55. Via a supply plug 57, the supply line 56 can be connected to a supply apparatus, not shown, for ener-gy/signal transmission, for example to the main unit 2 of the ventilation system 1.

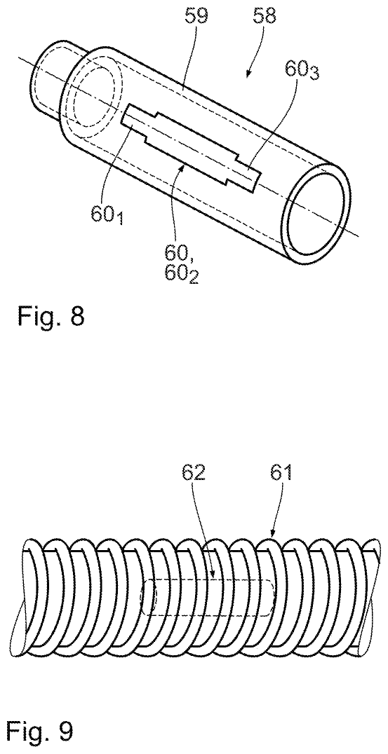

With reference to FIG. 8, a further embodiment of a monitoring connector 58 is described below. Components and functions corresponding to those already explained above with reference to FIGS. 1 to 7 bear the same reference numerals and will not be discussed again in detail.

In FIG. 8, a disposable of the monitoring connector 58 in the manner of the disposable 25 is shown. Alternatively, the monitoring connector 58 may be an integrated connector in the manner of the monitoring connector 50.

A portion 60 of a base body 59 of the monitoring connector 58, which forms a wall portion of the inner lumen 48 at the same time, is made of a thermochromic material. A color change sensitivity of the portion 60 is adapted to a predetermined operating temperature range of the breathing air to be conducted.

The portion 60 may be subdivided into a plurality of portions 60₁, 60₂, 60₃. These portions 60₁, 60₂, 60₃ may each be made of thermochromic materials having different color change temperatures. Alternatively or additionally, at least one of these portions 60₁, 60₂, 60₃ may be made of a reversible thermochromic material and at least one other of these portions 60₁, 60₂, 60₃ may be made of a non-reversible thermochromic material.

With reference to FIG. 9, a further embodiment of a tube component in the manner of the tube components 5, 11, 17, which have already been explained above in particular with reference to FIGS. 1 to 7, is described below. Components and functions corresponding to those already explained above with reference in particular to FIGS. 1 to 7 bear the same reference numerals and are not discussed again in detail.

In the case of the tube component 61 according to FIG. 9, a part of a tube wall is configured as a portion 62 made of thermochromic material. For the design of the portion 62 of the tube component 61, in particular in portions, what has been stated above regarding the portions 60; of the portion 60 according to FIG. 8 applies.

A basic component of the thermochromic material can be a thermoplastic elastomer or silicone.

In the case of the connectors 13 or 50 already described above, a component or a portion of a component which is thermally coupled to the conducted breathing air can also be made of thermochromic material.

The thermochromic material may be a material composition containing inorganic compounds of rutile and zinc oxide. Alternatively or additionally, components with bixanthylidene derivatives and/or bianthronylidene derivatives can be used. In principle, silver iodide compounds can also be used. Alternatively or additionally, bromothymol blue embedded in a pH-dependent polymer matrix can also be used. Further variants which can be components of the thermochromic material include a lithium chloride-containing polyether matrix, bis(diethylammonium) tetrachloridocu-prate(II) and salvatochromic dyes.

The ventilation system 1 can be used as follows:

In a deployment state, the monitoring connector 13 is present with the reusable 24 separated from the disposable 25, respectively. When the tube components 5, 11, and 17 are linked to each other and to the main unit 2 to make the ventilation system 1 ready for use, the reusable 24 is connected to the disposable 25 shortly before the first use begins, wherein the start signal is generated by the start signal generator unit 37 or 37, 43, as explained above. From the then determined start time, the microcontroller 37 in signal communication with the real time clock 39 senses the captured time period and thus monitors a duration of application of the disposable 25 during ventilation. For example, based on a maximum duration of application of seven days, the control/regulation unit 35 may control the light source 42 such that the light sources 42 emit green light during the first six and a half days. The remaining 12 hours until the maximum duration of application has been reached, the light may then switch from green to blue, which is controlled by the control/regulation unit 35. If the seven-day duration of application has been exceeded, a blue flashing signal may then be generated initially, which changes to a red signal after a grace period has elapsed, in each case again controlled by the control/regulation unit 35. In any case, when the maximum duration of application has been reached, the components of the ventilation system 1 that come into contact with the breathing air are replaced. The reusable 24 is then reused and connected to the new disposable 25 of the exchanged breathing air conducting components so that the duration-of-application cycle can start anew after the start signal has been generated by the start signal generator unit 37 or 37, 43.

The monitoring connector 13 can also be used for temperature measurement and, in particular, for temperature threshold detection. For this purpose, the breathing air temperature is detected via the sensor 45 and a corresponding temperature signal is transmitted to the microcontroller 37. As soon as the measured temperature exceeds a threshold value, for example 40° C., the control/regulation unit 35 controls the light sources 42 to output a visual warning signal, for example a red flashing light. As soon as, after exceeding the temperature threshold, the measured breathing air temperature falls below a second, defined lower temperature value, this visual indication can be terminated again, controlled by the control/regulation unit 35.

The memory units as part of the microcontroller 37 or 43 can also be used to store a digital business card of the monitoring connector 13 as a whole or also of the reusable 24 or the disposable 25. The identification data set, a type designation of the monitoring connector 13 as well as of the entire ventilation system 1, an instruction manual for the monitoring connector and/or the ventilation system as well as further data, for example for rele-vant usage data, may be part of a corresponding business card data set. The identification data set can be designed as plagiarism protection. As usage data in the memory of the microcontroller 37, for example, the IDs of the disposables 25 that have come into use over the service life of the reusable 24 can be stored, or also measurement data progressions of the sensor 45 over time, as well as connector states detected by means of the con-trol/regulation unit 35 via the actuation sequences of the light signals 2, in particular alarm events (maximum duration of use exceeded/temperature threshold exceeded).

The sensor 45 can be embodied with an integrated EEPROM. This can be used in particular to specify temperature threshold values by programming. In the memory of the sensor 45, an individual identification data set of the disposable 25 may be stored as well, which can be provided there when the disposable 25 is produced. Alternatively, such an identification data set may be written to the memory of the sensor 45 by the microcontroller 37 of the reusable 24 when these two components are electrically connected or even when the duration of use is started.

The control unit 35 can be in signal communication with an external display unit, for example with a graphical user interface (GUI), via a wireless or wired interface. This can be used, for example, to display the instantane-ous temperature or to display the current duration of use and further sensor data.

A log file may be written in the memory component 43 or in the memory component of the microcontroller 37, which logs the status parameters of the monitoring connector 13.

Alternatively or in addition to the temperature sensor 45, a humidity sensor, a mass flow or flow sensor, a sensor for determining a condensation water agglomeration or even a gas analysis sensor for determining a gas composition of the breathing air and, in particular, for monitoring a con-tamination and/or an exceeding of critical component threshold values may be used.

Provided that a breathing air pressure sensor is used in the monitoring connector 13, it can be used to regulate a breathing effort adjustment.

In addition, a pressure or strain sensor can be used to detect a mechanical load on the monitoring connector 13. Such a sensor can have a membrane structure and/or a meander structure. A pressure or force application leads to a structural strain in such a pressure or strain sensor, which can be detected and converted into pressure and/or force values. Such a pressure sensor may be arranged in a portion of the monitoring connector 13, e.g. in the reusable 24 or the disposable 25. Alternatively or additionally, such a pressure or strain sensor can be arranged as an external sensor, for example in the breathing air tube component 11, and be in signal communication with the monitoring connector 13 via corresponding interfaces.

Such a pressure or strain sensor can alternatively or additionally monitor a pressure load on the tube components 5, 11 or 17. In this way, it is possible to detect an undesirable load on one of these components, e.g. a kinking or bending of the breathing air tube component 5, 11 or 17, of the ventilation system 1 in order to quickly take countermeasures by visual or audio alarm, if necessary.

Further sensors that can be used in the ventilation system 1 either integrated in the monitoring connector or as external sensors include a sensor for measuring the patient's skin temperature, a sensor for measuring the patient's skin moisture, a sensor for measuring the patient's skin color, a heart rate sensor, an oxygen saturation sensor, a gas analysis sensor, a fire safety sensor, an orientation sensor for determining a position of at least one component of the ventilation system 1 in space, a motion sensor for determining a movement of at least one component of the ventilation system 1, a sensor for monitoring a drug dispensing.

Processing, in particular preprocessing, of the sensor data transmitted to the microcontroller 37 may be performed in the microcontroller 37. For example, the data may be averaged or filtered or compressed to reduce memory requirements. This (pre-)processed data may then be forwarded, for example, the main unit 2 and/or to another external data processing component for external further processing or display.

The components of the ventilation system 1 that conduct breathing air may have an antimicrobial coating.

A gas flow measurement can be used to infer a leakage in the ventilation system 1, in particular by appropriate processing in the microcontroller 37 and/or in the main unit 2.

The microcontroller 37 may be designed to be programmable. In addition, the memory of the microcontroller 37 may include a program library from which programs or program components may be selected.

The reusable 24 can also have further wireless interfaces for reading out, in particular, recorded measurement data, for example via RFID, via NFC, via Bluetooth or via WLAN.

Via one of the interfaces described above, the control/regulation unit 35 can be in signal communication with at least one sensor external to the connector 13, 50 or 58. As an example of such a sensor, FIG. 1 schematically shows such a sensor at 63, which may be a sensor for measuring a heart rate of the patient, a sensor for measuring a body temperature of the patient, and/or a sensor for measuring a skin surface resistance of the patient.

1. A connector (13; 50; 58) for a patient ventilation system
to connect to a heatable breathing air tube portion (11) for conducting ventilation air from a breathing air source (2) to a patient,
to connect the breathing air tube portion (11) to a patient air interface (15),
wherein at least one portion (60; 60₁, 60₂; 62₃) of the connector (13; 50; 58) is made of a thermochromic material,
wherein the thermochromic material includes a plastic matrix having a poly(organo)siloxane.

2. A connector (58) for a patient ventilation system
to connect to a heatable breathing air tube portion (11) for conducting ventilation air from a breathing air source (2) to a patient,
to connect the breathing air tube portion (11) to a patient air interface (15),
wherein at least one portion (60; 60₁, 60₂; 62₃) of the connector (58) is made of a thermochromic material,
wherein the connector (58) has a plurality of portions (60₁, 60₂, 60₃) which are each made of a thermochromic material having different color change temperatures.

3. The connector according to claim 2, wherein at least one of the portions (60₁, 60₂, 60₃) is made of a reversible thermochromic material and that at least another one of the portions (60₁, 60₂, 60₃) is made of a non-reversible thermochromic material.

4. An assembly having at least one connector according to claim 1, and a breathing air tube component (11) for conducting breathing air.

5. The assembly according to claim 4, wherein at least one tube portion (62) of the breathing air tube component is in contact with or thermally coupled to the ventilation air, wherein the at least one tube portion (62) is made of a thermochromic material.
